(19) United States
(12) Patent Application Publication
(10) Pub. No.: US 2016/0228287 A1
(43) Pub. Date: Aug. 11, 2016

(54) SYSTEMS AND METHODS FOR PERMANENT FEMALE CONTRACEPTION

(71) Applicant: Cirrus Technologies KFT, Budapest (HU)

(72) Inventors: John H. SHADDUCK, Menlo Park, CA (US); Csaba TRUCKAI, Saratoga, CA (US)

(73) Assignee: Cirrus Technologies KFT, Budapest (HU)

(21) Appl. No.: 15/018,725

(22) Filed: Feb. 8, 2016

Related U.S. Application Data
(60) Provisional application No. 62/113,321, filed on Feb. 6, 2015.

(51) Int. Cl. A61F 6/22

(52) U.S. Cl. CPC A61F 6/225

(57) ABSTRACT

Devices and methods for accessing a female patient's fallopian tubes.

### CROSS-REFERENCE To RELATED APPLICATIONS

This application claims benefit of priority to U.S. Provisional Application No. 62/113,321, filed Feb. 6, 2015, the content of which is incorporated by reference herein in its entirety.

### FIELD OF THE INVENTION

The present invention relates to medical devices and methods for treating and occluding a female patient's fallopian tubes to provide birth control or sterilization where the duration can be long term or permanent.

### BACKGROUND

Female sterilization typically involves occluding a patient's fallopian tubes, with various procedures using laparoscopic or minimally invasive trans-cervical approaches. One procedure involves placing flexible coil-like devices into the fallopian tithes which are made of polyester fibers and metal wires. Tissue in-growth into the implanted devices can block the fallopian tubes. However, such implants are worrisome due to potential unknown long term effects.

### SUMMARY OF THE INVENTION

The present invention is directed to catheter systems and implants together with methods of using such systems and device for occluding reproductive body lumens such as a female's fallopian tubes.

The present disclosure includes methods and devices for accessing a fallopian tube. In one variation, the method includes trans-cervically introducing an elongate device into a patient's uterine cavity, the elongate device including, a flexible guide sleeve having a guide channel with an open distal end; expanding an expandable member in the uterine cavity to open the uterine cavity and align the guide channel with a fallopian tube.

The method can include inflating the expandable member with a liquid or gas. In some variations, the expandable member is shaped to conform to the anatomy, such as the triangular shape of the uterine cavity. In such a case, the expandable member has a triangular shape and the distal termination is proximate an apex of said triangular shape.

The method can include advancing a catheter through the guide channel and into the fallopian tube. Another variation also includes that the open distal end of the guide channel opens on a first lateral side of the expandable member. In additional variations, the expandable member comprises a triangular shape and the guide channel opens on a distal apex of the triangular shape.

In another variation, the methods and devices can include a second flexible guide sleeve having a second guide channel with a second open distal end, where the second open distal end of the second guide channel opens on a second lateral side of the expandable member that is opposite to the first lateral side of the expandable member.

The method can also include adjusting an alignment of the guide channel by deflecting an orientation of the flexible guide sleeve within the expandable member.

In another example, the devices described herein to access a fallopian tube can comprise an expandable member comprising a triangular shape having a distal base with a first apex and a second apex on either end of the distal base, and a proximal base opposite to the distal base; a flexible guide sleeve having a passageway extending therethrough, the flexible guide sleeve extending through the expandable member from the proximal base through to the first apex along the distal base such that the passageway opens at the first apex on a lateral side of the distal base.

Variations of the device can further comprise an external sleeve exterior to the flexible guide sleeve where the external sleeve and flexible guide sleeve are moveable relative to each other.

In an additional variation, the access device can include a distal end of the flexible guide sleeve that is affixed to the expandable member such that a profile of the flexible guide sleeve within the expandable member can be adjusted h relative movement of the flexible guide sleeve to the expandable member.

In a further variation, the device can include a second flexible guide sleeve having a second passageway that opens at a second apex on a side of the distal base opposite to the first apex

The tubal occlusion procedure described herein can be a minimally invasive procedure in which a device is introduced into the patient's uterine cavity trans-cervically. In one aspect RF energy is used to ablate a thin layer of tissue in a segment of a fallopian tube which can be performed very rapidly, for example in 5 to 60 seconds. A second step of the method involves cutting or damaging tissue within the segment to cause bleeding and a subsequent adhesion formation across the coagulated blood. The wound healing response and adhesion of the walls in the segment can close the fallopian tube. The occlusion caused by the wound healing response can be permanent or have an extended duration in which passage through the segment is blocked.

The details of one or more embodiments of the invention are set forth in the accompanying drawings and the description below. Other features, objects, and advantages of the invention will be apparent from the description and drawings, and from the claims.

The features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention

### BRIEF DESCRIPTION OF THE DRAWINGS

FIG. 1A is a sectional view of a patients uterus and fallopian tubes showing a system of the invention for occluding a fallopian tube, wherein the system includes a catheter carrying an implant and FIG. 1A illustrates an initial step in a method corresponding to the invention wherein a hysteroscope is introduced transcervically into the uterine cavity and the catheter is advanced toward the opening of a fallopian tube.

FIG. 1B is an enlarged view of a portion of the uterus and fallopian tube of FIG. 1A illustrating another step in a method of the invention wherein a guidewire is advanced through the catheter and into the fallopian tube.

FIG. 1C is a view similar to that of FIG. 1B illustrating another step in the method wherein the catheter and implant are advanced over the guidewire to a targeted site in the fallopian tube.

FIG. 1D is a view similar to that of FIG. 1C illustrating another step in the method wherein a retaining sleeve carried by the catheter is retracted to expose the implant in the targeted site in the fallopian tube, and FIG. 1C also illustrates a subsequent step of delivering ablative energy to walls of the fallopian, and another step of causing bleeding in the site as further shown in FIG. 2A.

FIG. 1E is a view similar to that of FIG. 1D illustrating another step in the method wherein the guidewire is withdrawn from the implant and the resilient implant moves to its non-tensioned configuration to flatten the fallopian tube.

FIG. 2A is an isometric view of an occluding device or implant carries by the catheter of FIGS. 1A-1D, with the implant body being maintained in a tensioned linear shape by the guidewire in a passageway of the implant, with FIG. 2A further illustrating a blade element that can be extended from the implant to cause bleeding in the targeted site in the step of FIG. 1D.

FIG. 2B is another view of the implant of FIG. 2A with the implant body in a non-tensioned shape having multiple curves with the guidewire withdrawn from the implant, and further illustrating the blade element extended from the implant for causing bleeding in the targeted site, for example, in the step of FIG. 1E.

FIG. 3 is a graphic representation of the fallopian tube with the tube walls approximated which corresponds to the method step shown in FIG. 1E.

FIG. 4A is a sectional view of the fallopian tube of FIG. 3 taken along line 4A-4A which again corresponds to the method step shown in FIG. 1F wherein blood accumulates and is trapped in the fallopian tube.

FIG. 4B is a sectional similar to that of FIG. 4A after the passage of time wherein an adhesion has formed across the lumen of the fallopian tube and further depicting the bin-absorption of the implant body.

FIG. 5 is a perspective view of another variation of occluding device or implant that includes the functionality of the system and implant of FIGS. 1A-4B.

FIG. 6A is a perspective view of another variation of occluding device or implant in a collapsed or non-extended position.

FIG. 6B is a view of the implant of FIG. 6A in an extended position.

FIG. 7A is a perspective view of another variation of occluding device or implant in collapsed or non-extended position.

FIG. 7B is a view of the implant of FIG. 7A in an extended position.

FIG. 8 is a perspective view of another variation of occluding device or implant in an actuated position.

FIG. 9A is a perspective view of another variation of occluding device or implant in an insertion configuration.

FIG. 98 is a view of the implant of FIG. 9A is a deployed configuration.

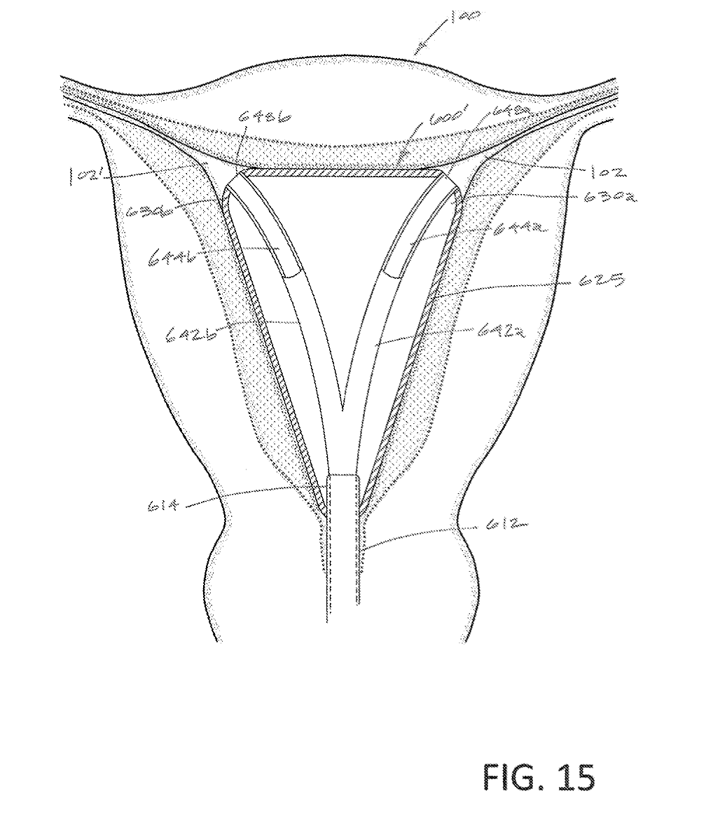

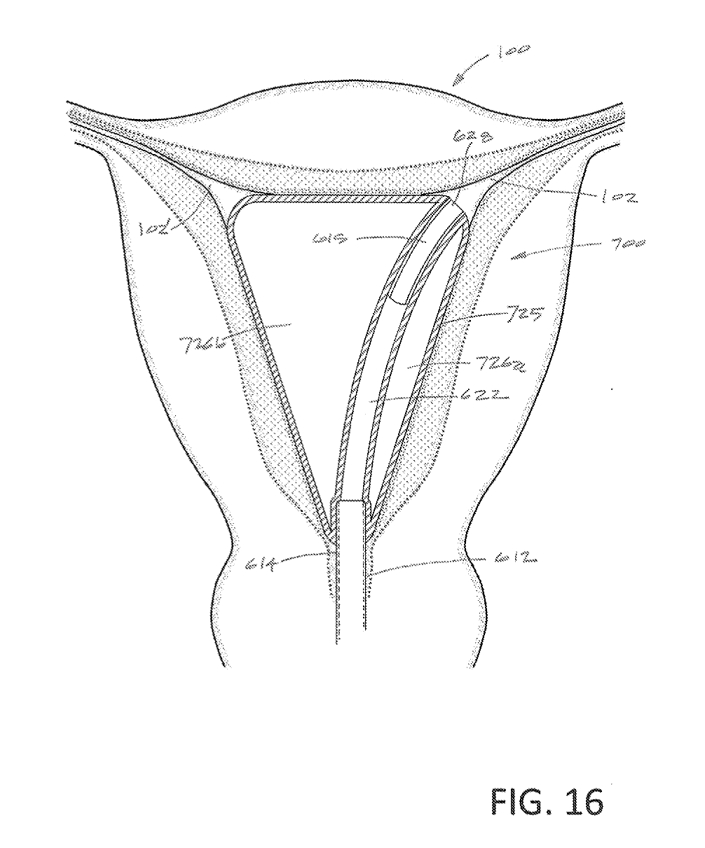

FIG. 9C is a view of the implant of FIG. 9B deployed in a fallopian tube to thereby flatten the tube.

FIG. 10A is a perspective view of another variation of occluding device or implant in an insertion configuration.

FIG. 10B is a view of the implant of FIG. 10A is a deployed configuration.

FIG. 11A is a perspective view of another variation of implant in an insertion configuration.

FIG. 11B is a view of the implant of FIG. 11A is a deployed configuration.

FIG. 12A is a perspective view of another variation of implant in an insertion configuration.

FIG. 12B is a view of the implant of FIG. 12A is a deployed configuration.

FIG. 13A illustrates accessing a fallopian tithe wherein an introducer and guide sleeve are advanced trans-cervically into the patient's uterine cavity

FIG. 13B illustrates a subsequent procedure to that of FIG. 13A wherein the introducer sleeve is retracted and the exposed expandable structure in then expanded.

FIG. 14 is a cut-away view of the expandable structure of FIG. 13B showing advancement of an articulating endoscope and treatment catheter through the guide sleeve.

FIG. 15 is a cut-away view of another variation of the access device with first and second guide sleeves carried within an expandable structure for accessing both fallopian tubes.

FIG. 16 is a cut-away view of another variation of the access device with multiple inflation chambers in an expandable structure for adjusting the orientation of the guide sleeve.

### DETAILED DESCRIPTION THE INVENTION

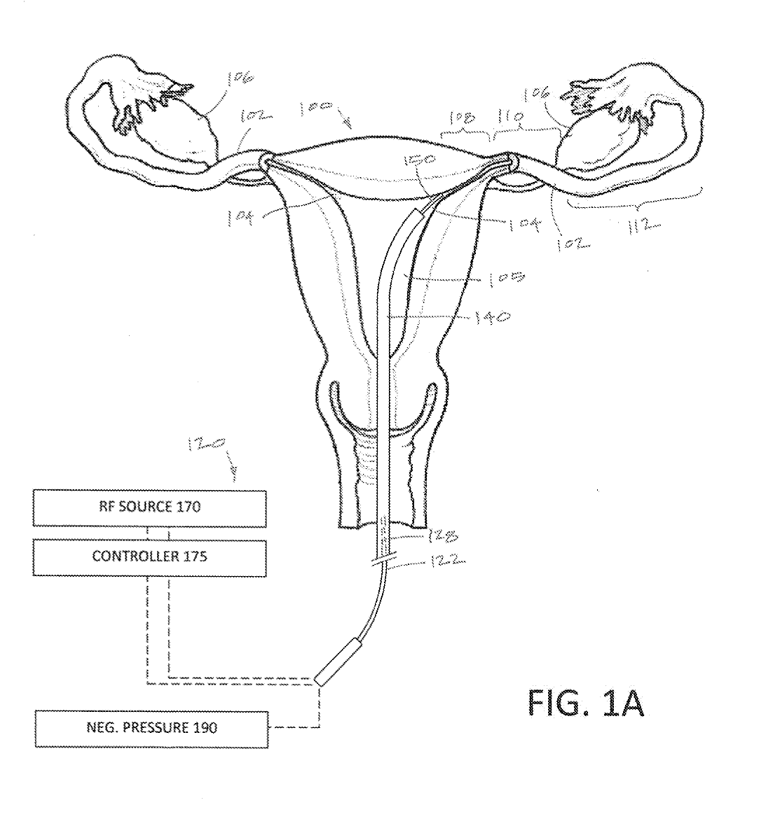

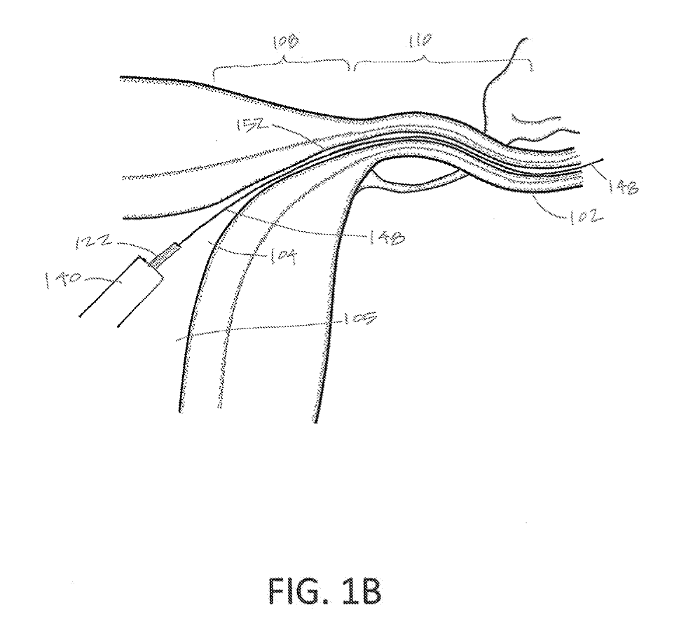

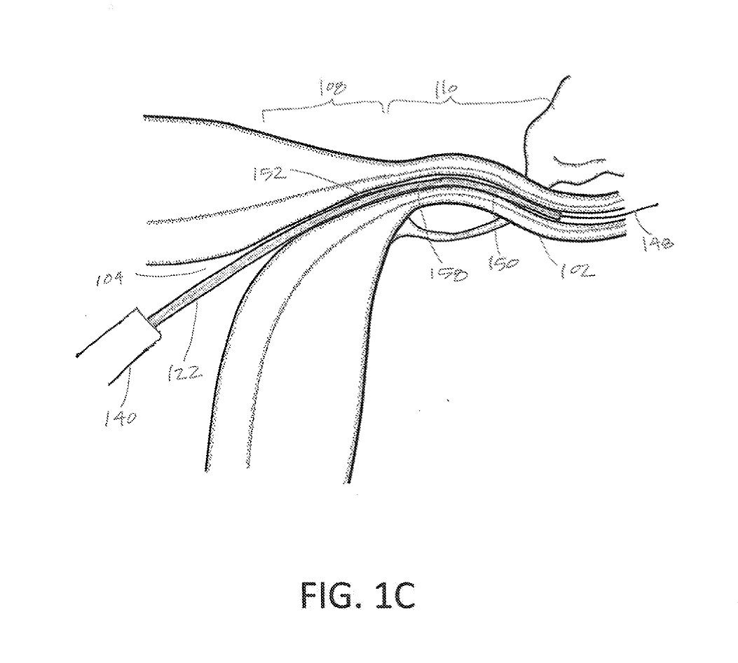

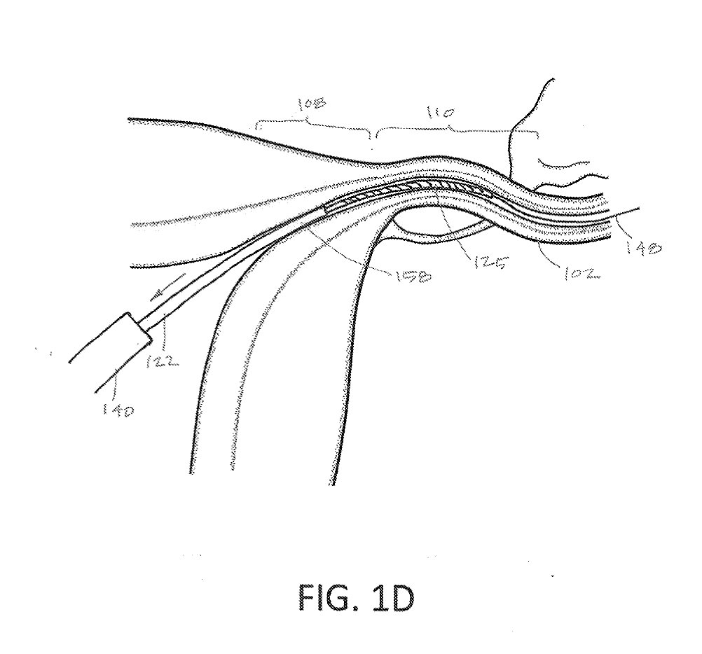

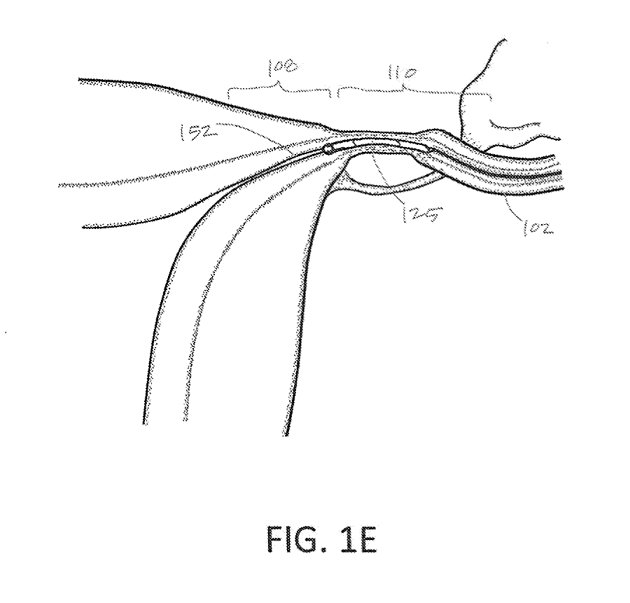

FIG. 1A illustrates a patient's uterus 100 and fallopian tubes 102 or oviducts, which are paired, tubular conduits that extend from the cornua 104 of the uterine cavity 105 us toward the ovaries 106. Each fallopian is about 7 cm to 14 cm in length and is defined by three different sections: the intramural segment 108, the isthmus segment 110 and the ampulla 112 (FIGS. 1A-1B). The intramural or interstitial segment 108 of the tube continues from the corium 104 to the isthmus 110 and is about 1 cm in length with a 1 mm lumen diameter. The isthmus 108 is a round cord-like structure which constitutes the medial one-third of the fallopian tube with a 2 mm to 10 mm outer diameter. The lumen of the fallopian tube is lined with a layer of mucous membrane that can have many folds and papillae. The wall of the fallopian tube includes layers of muscle tissue. The innermost layer has spirally arranged fibers, the middle layer has circular fibers, and an outer layer has longitudinal muscle fibers. These muscle fibers provide for peristalsis and antiperistalsis in the fallopian tubes.

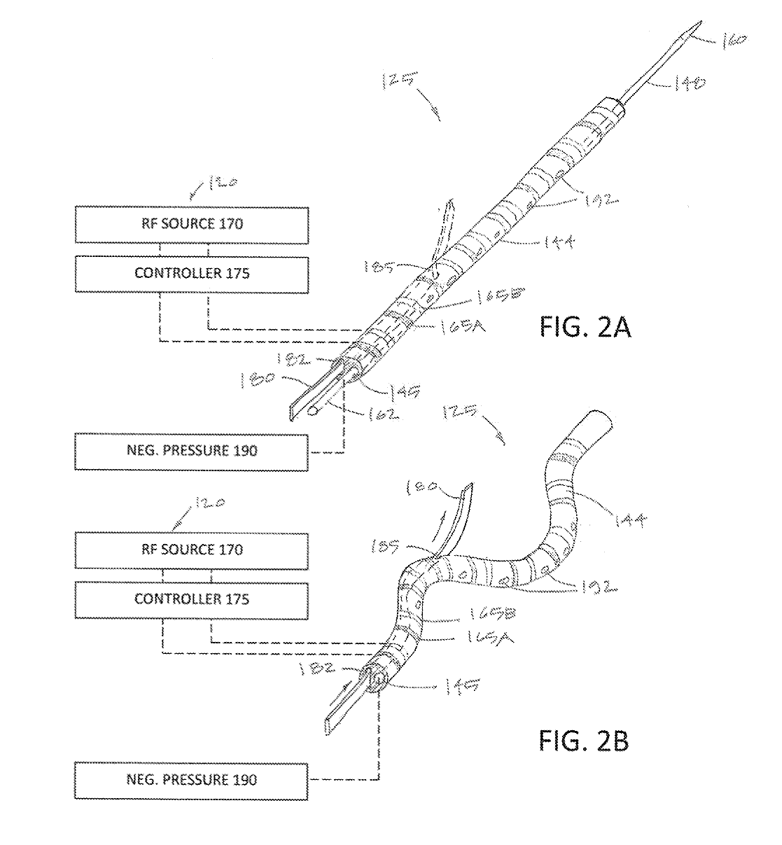

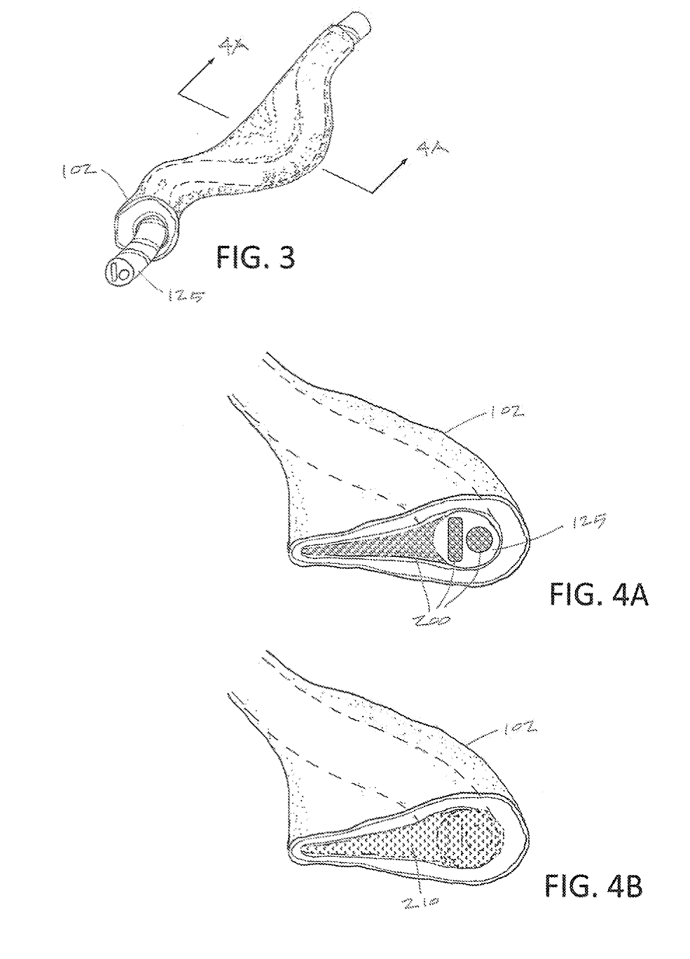

FIGS. 1A-1E and 2A-2B illustrate a system 120 that includes an elongate catheter 122 that carries a releasable occluding device or implant 125 (FIG. 2A) which is adapted to occlude a patient's reproductive lumen such as fallopian tube 102. The catheter 122 can have any suitable length for extending through the working, channel 128 of a hysteroscope or endoscope 140. In one embodiment shown in FIGS. 1A-1D, the hysteroscope 140 is an articulating scope that can be articulated in the uterine cavity 105 to view the entry to the fallopian tubes 102 and direct the catheter into a fallopian tube 102. In another variation, a straight rigid endoscope could be used with an appropriate viewing angle of 5° to 30° together with a catheter or catheter sleeve that can be articulated to assist in directing a catheter working end into a fallopian tube.

In one variation of implant 125 shown in FIGS. 2A-2B, the body 144 of the implant comprises a polymeric material with a passageway 145 to allow it advancement over a guidewire 148. In general, the variations of catheter working end 150 and implant 125 disclosed herein are adapted to provide functionality in more than one aspect which thus enables the system to effectively occlude fallopian tubes to provide permanent contraception. In one aspect and function, the system and/or implant provide a mechanism to deliver energy to the catheter working end or implant to ablate tissue in the fallopian tube lumen 152 over an elongated segment. As will be described further below, the ablation of endothelial tissue over an elongated segment prevents that rapid re-epithelialization of the lumen, and ablation of underlying muscle layers prevents peristalsis which otherwise could move or disrupt coagulum described next. In a second aspect, the system and/or implant provide means for causing bleeding with a targeted segment of a fallopian tube. As will be described further below, bleeding and coagulum at the targeted site will optimize conditions for fibrosis and adhesion formation in the targeted site for permanent occlusion. In a third aspect, as will be described further below, the implant 150 provides a ‘dam ’ for preventing displacement of the coagulum following bleeding to allow time for the adhesion to fully develop across to coagulum. In a fourth aspect, as will be described further below, the implant 150 provides a means for approximating fallopian tube walls to lessen or eliminate the adhesion dimension between the walls to accelerate the time required for adhesion formation. In a fifth aspect, as will be described further below, the implant 150 had a very flexible body 144 to allow its insertion into a tortuous path of a fallopian tube over a flexible guidewire. In a sixth aspect, as will be described further below, the implant 125 provides a means for resisting movement of the implant within the fallopian tube 102 which can be the overall shape of the implant or barb-like features on the implant or adhesives carried by the implant for engaging tissue. In a seventh aspect, as will be described further below, the implant 125 can be fabricated at least partly of micro-porous polymeric material that allows for tissue in-growth in a scaffold-like implant body. In an eighth aspect, as will be described further below, the implant 125 can be fabricated at least partly of bio-absorbable or bio-degradable material which will lessen its bulk following absorption or degradation.

FIGS. 1A-1F provide an overview of the steps in a method corresponding to the invention, and further functional details of the system 120 and implant 125 in each of the steps follow this overview.

In FIG. 1A, an articulating hysteroscope 140 is introduced transcervically and articulated to view in the direction of a fallopian tube 102. The catheter 122 together with a guidewire 148 is then introduced through the working channel 128 of the hysteroscope.

FIG. 18 illustrates a subsequent step wherein the physician introduces guidewire 148 into and through the lumen of the fallopian tube 102 to at least the isthmus segment 110. FIG. IC then shows another step in which the catheter working end 150 is advanced over the guidewire 148 into the fallopian tube 102.

FIG. 1C illustrates one embodiment of implant 125 which is carried by the catheter working end within a thin-wall sheath 158 that can be retracted to expose the implant 125.

FIG. 1D next shows another step in which the sheath 158 is retracted to expose and deploy the implant 125 in the intramural and or isthmus segment of the fallopian tube 102. At this step, the system and implant can be actuated to cause bleeding in the targeted segment of the fallopian tube. Also at this step, the implant 125 is still operatively coupled to the catheter to allow energy delivery from a remote energy source to the implant as will be described below.

FIG. 1E shows the implant 125 in the fallopian tube after being de-coupled from the catheter. As will be described below, the implant when released from the catheter moves from a first more linear shape to a second non-linear shape which is adapted to flatten the fallopian tube to thereby approximate walls of the fallopian tube.

FIG. 1F illustrates the implant 125 in portion of the fallopian tube in its second non-linear shape approximating the walls of the fallopian tube 102.

FIGS. 1G and 1H depict a portion of the fallopian tube segment following approximation o the walls with the pooling of blood and resulting coagulum in the targeted site, followed by adhesion formation in the site and bio-absorption of the body of the implant 125.

Now turning, to FIGS. 2A-2B, the implant 125 can be described in more detail, The implant body 144 can be fabricated of a polymeric material that is flexible or the polymer can be more rigid and formed as a slotted tube as is known in the art to provide flexibility. In one variation, the implant can have a diameter ranging between 1 mm to 3 mm with a length ranging between 1 cm to 3 cm. In the variation shown in FIGS. 2A-2B, the implant has a passageway 145 to allow it to be advanced over guidewire 148. The guidewire 148 can have a highly flexible tip portion 160 adapted for negotiating through a tortuous path of a fallopian tube and a stiffer portion 162 proximal to the highly flexible portion that can function to straighten the fallopian tube and also maintain the implant in a suitable linear shape as in 2B. In the variation of FIGS. 2A-2B, the implant 125 can be maintained in a tensioned shape by guidewire 148 as shown in FIG. 2A which allows for introduction into the fallopian as shown in FIGS. 1C and 1D.

FIGS. 2A-2B further illustrate an energy delivery component of the system wherein the implant 125 carries opposing polarity bi-polar electrodes 165A and 165B that are operatively coupled to RF source 170 and controller 175. The spaced apart electrodes 165A and 165B are shown in FIGS. 2A-2B in a helical configuration over the length of the implant but it should be appreciated that such electrodes can have any form or pattern, including circular, linear, dotted, fragmented or concentric in an outer implant surface an inner passageway of the implant. In operation, the RF source can be actuated at a suitable power level for about 5 seconds to 1 minute to ablate tissue in the fallopian tube lumen. In one variation, the mucosal layer is ablated over the length of the implant which can be from 1 cm to 3 cm. In this variation, the duty cycle of RF energy delivery can further ablate the underlying circular, longitudinal, and spiral muscle layers, which can be a depth of about 0.25 mm to 1 mm. The ablation of the muscle fibers over an elongated segment then will prevent peristalsis and antiperistalsis and thereby assist in preventing displacement of the implant 125 and blood and/or coagulum. The ablation step typically would be performed with the implant in its tensioned shape with the guidewire straightening the implant. In another variation of the method, the ablation step could be performed following withdrawal of the guidewire 148 with the implant 125 in it non-tensioned configuration. The implant 125 can be a resilient polymer that is pre-formed in a curved or sinuous shape, wherein the inherent spring-ability of the implant body will urge it toward its non-tensioned curved shape. In another variation, the implant's resiliency to urge its shape toward its curved shape of FIG. 2B also be assisted by a metal spring element embedded in the implant body 144. The implant can have any curved shape that can include 1-10 or more curves or a similar number of angled portions with living hinges. In one variation the curved or angled portions are configured to provide a flat or planar shape when the implant is in its non-tensioned position to flatten the fallopian tube 102 to thereby approximate the walls of the tube.

FIGS. 2A-2B further illustrate a mechanism carried by the catheter and implant 125 that can be actuated to cause bleeding at the site. In one variation shown in FIG. 2A, it can be seen that a thin flexible blade 180, for example of ribbon stainless steel as used in razor blade, can be moved axially in slot 182 that extends through the catheter and implant 125 to exit an open slot termination 185 to pierce and cut tissue. The blade 180 can be extended from open termination 185 an extension distance of 1 mm to 5 mm, and usually from 1 mm to 2 mm. In any event, the depth of penetration of blade 180 into tissue is greater than the depth of the ablation to insure bleeding through any ablated layer, in use, with reference to the method steps of FIGS. 1D and 1E, the catheter and implant 125 can be rotated in either direction, and at various degrees of rotation, the blade 180 can be extended and retracted to cut tissue and cause bleeding. In use, the blade 180 can be extended following the ablation step with the implant 125 in either its tensioned configuration (FIGS. 1D and 2A) or non-tensioned configuration (FIGS. 1E and 2B).

In another aspect of the method step shown in FIGS. 1D and 1E, a negative pressure source 190 can be actuated contemporaneous with or subsequent to the cutting step to draw blood from the cut tissue into the site. As can be understood from FIGS. 2A-2B, the negative pressure source 190 can be actuated manually or by controller 175 in unison with the ablative energy, or automatically timed to follow the actuation of ablative energy. The negative pressure or suction can communicate with the targeted site through the guidewire passageway 148 in the catheter and implant 125, and/or the slot 182 for blade 180 that extends through the catheter and implant. In FIGS. 2A-2B, the guidewire passageway 148 communicates with the negative pressure source 190 to thereby apply suction forces through a plurality of ports 192 in the implant 125. In one variation, the suction forces are pulsed to sustain bleeding into the site. FIGS. 1F-1G show that the blade 180 along with the guidewire 148 can be withdrawn from the implant 125.

In one variation, the implant 125 is releasably carries by the catheter within the retractable sheath 158. Thus, after the sheath is withdrawn as illustrated in FIG. 1D, the implant 125 is free from the catheter shaft but still stabilized in place by the guidewire 148. In other variation, the implant can be released from the catheter shaft by means known in the art, such as (i) a tear-away connection that is broken by retraction of the guidewire 148 or blade 180, (ii) a mechanical mechanism such as a latching collar; (iii) a meltable polymer connection that can be melted by RF or resistive heating; (iv) a frangible connector actuated and broken by a heated NiTi element; or (v) an electrolytic detaching mechanism as known in the art of detachable embolic coils.

Now turning to FIGS. 1F-1H, it can be seen how the implant 125 is adapted to trap blood 200 and coagulum in the site. In FIG. 1F, the guide wire has been withdrawn and the implant 125 is urged toward its non-tensioned shape to flatten the fallopian tube 102 wherein the approximated walls of the fallopian tube 102 will allow for more rapid adhesion formation between the opposing walls as shown in FIG. 1G.

FIG. 1G illustrates blood 200 pooling in the flattened segment of the fallopian tube 102. The blood also migrates into the guidewire passageway 148 through ports 192 and into the blade slot 182 through open termination 185.

Of particular interest, it can be understood from FIG. 1E that the curved shape of implant 125 will help lock it in place in the fallopian tube 102 to resist any peristaltic forces that might otherwise dislodge the implant. Also of particular interest, the curve or curves of the implant body as shown in FIG. 1F are adapted to function as a dam to prevent the blood and subsequent coagulum from being displaced.

FIG. 1H illustrates the fallopian tube 102 being occluded with adhesion 210 which can form rapidly in a few days as the trapped blood/coagulum (FIG. 1G) functions as an optimal scaffold for fibrosis across and between the walls of the fallopian tube 102. FIGS. 1G-1H also show the flattening of the fallopian tube 102 which allows a more rapid formation of the adhesion 110 due to the reduced thickness dimension between the approximated walls of the fallopian tube 102.

FIG. 1H also is a graphic representation of one variation of the device and method wherein the implant 125 is bio-absorbable and FIG. 1H illustrates that the implant 125 has been resorbed and replaced with the adhesion 110.

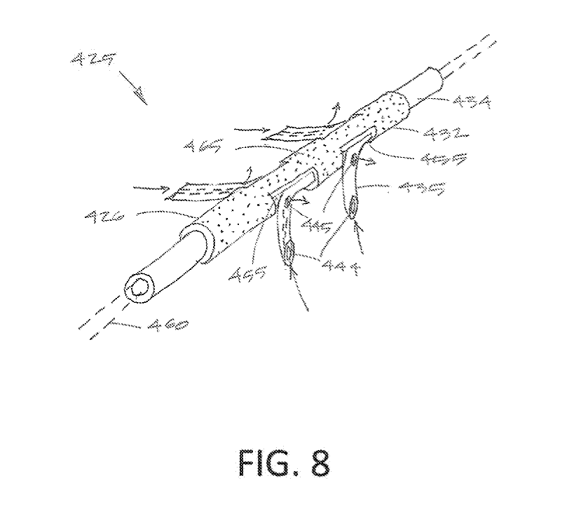

FIG. 5 illustrates another variation of implant 225 that can be used to occlude a fallopian tube using, in general, the same methods as described in FIGS. 1A-4B. The body 226 of implant 225 can comprise a slotted polymer tube having interior lumen 228 in which the slots 240 can have selected dimensions to allow a rigid polymer tube to be flexible to follow a guidewire 248 within a tortuous path. The slots can be formed to provide flexibility in 360° as is known in the art. In this respect, the polymer sleeve can comprise a bio-absorbable or bio-degradable material that is substantially rigid but made flexible by the slots 240.

Still referring to FIG. 5, the ablation functionality of the implant can again be provided by an RF source and spaced apart opposing polarity electrodes can be printed on the surface of the implant body 226. In another variation, the surface of the implant body 226 can have electroless plating of gold or another conductive metal to provide a first electrode and the guidewire 248 can comprise a second opposing polarity electrode.

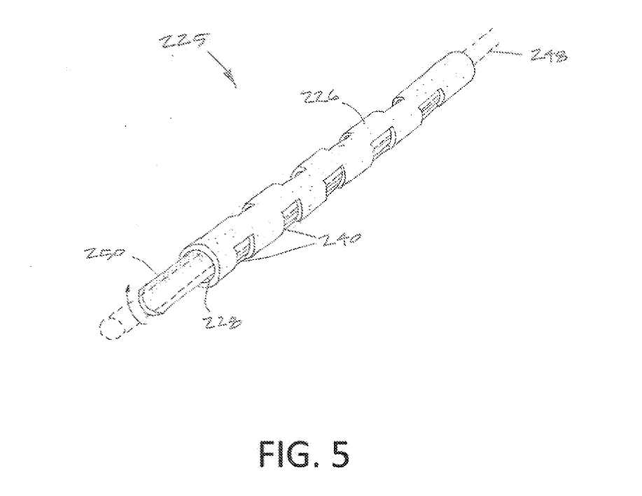

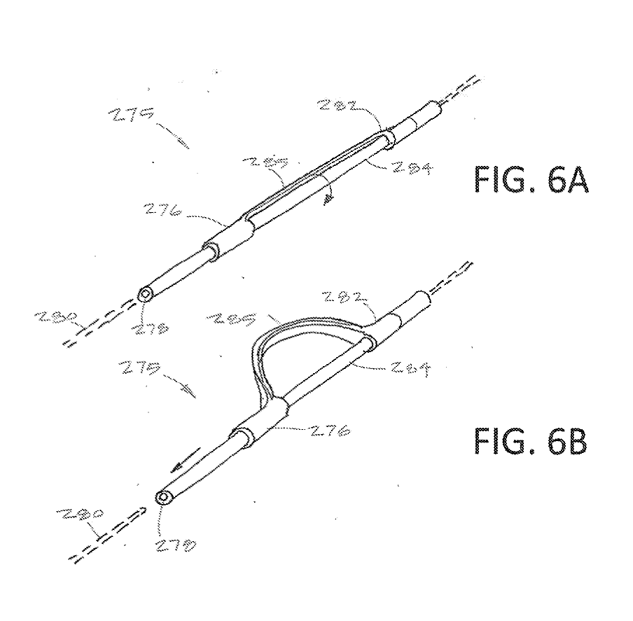

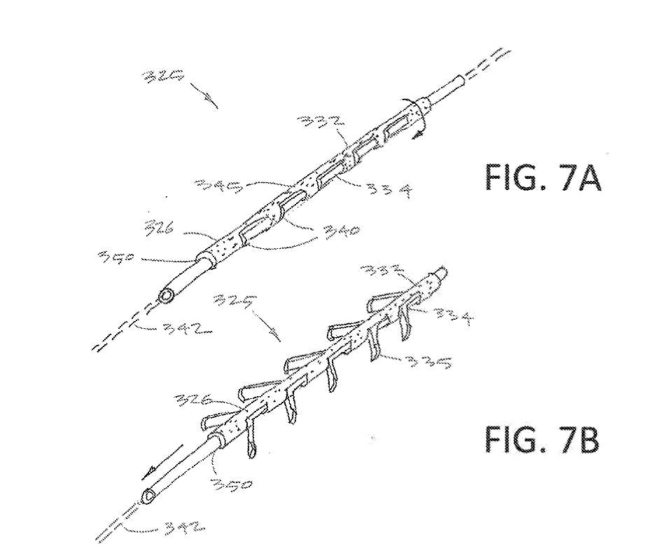

Still referring to FIG. 5, the mechanism to cause bleeding associated with the implant 225 comprises a cutting element or blade 250 that extends through lumen 228 and can be actuated from the handle of the catheter and can be manually operated or motor driven. The blade 250 can be a rotatable thin linear member of a ribbon stainless steel as shown in FIG. 5, but also can be a helical sharp edged element or an abrasive wire that can be moved rotational, axially or in both rotational and axial directions. An additional advantage of the variation of FIG. 5 is that the negative pressure source 190 can suction tissue into lumen 228 and the tissue can be cut and captured in the lumen 228. The cutting depth is sufficient to cut through the ablated tissue layer. The implant 225 can be moved slightly both axially and rotationally while actuating the blade to resect the entire surface layers of the fallopian tube lumen 152. As a result, bleeding, is caused and further, the approximated walls or the fallopian tube 102 will be raw tissue, instead of ablated layers with cuts therein as shown in the embodiment in FIGS. 1A-4B. It is believed that adhesions will form more quickly with the exposed cut tissue interfacing the coagulum in the targeted site (cf. FIGS. 3-4B).

Still referring to FIG. 5, the implant 225 can flatten the fallopian tube by providing a pull wire in the side of the sleeve to cause a curve in the implant (not shown). In another variation, a heat shrink polymer can be provided on one side of the implant that can be heated to deform the implant. Thus, the implant 225 of FIG. 5 can provide all the functions as described in the previous embodiment, including: flexibility to follow a tortuous path, an RF electrode arrangement to ablate tissue, a cutting mechanism to cause bleeding in a targeted site, means to flatten the fallopian tube and means to trap the coagulum in the targeted site.

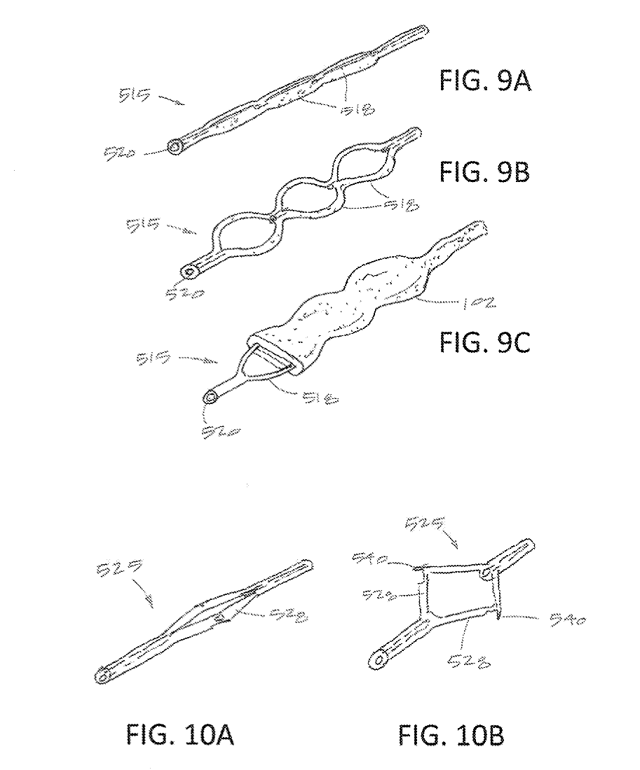

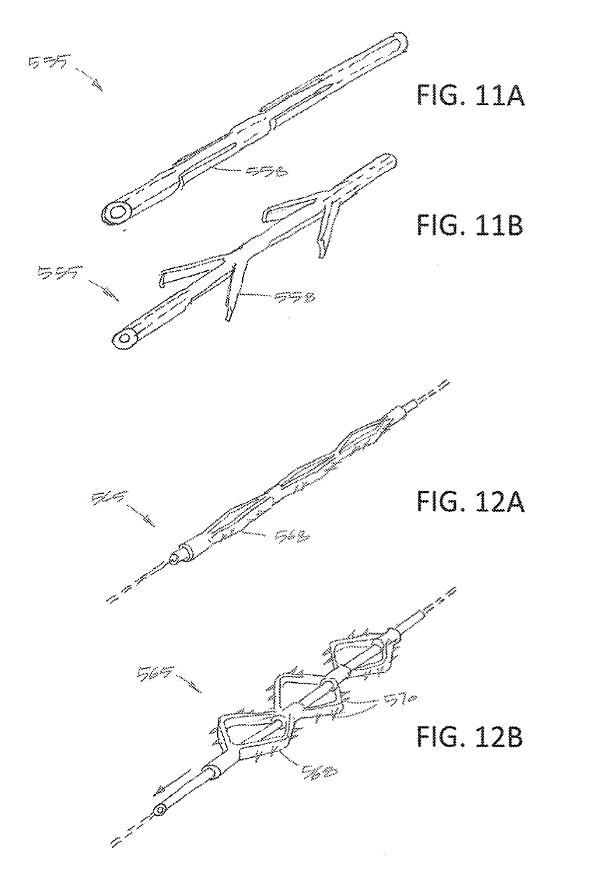

FIGS. 6A-6B illustrate another variation of implant 275 for occluding a fallopian tube that can function to perform the methods as described previously. The body 276 of implant 275 again can comprise polymers with a guidewire lumen 278 to accommodate guidewire 280. The implant has first and second (outer and inner) elements 282 and 284 that can be actuated to flatten the fallopian tube lumen. The outer element 282 has a flexible medial section that carries an abrasive edge 285 for example of diamond powder. Thus, the outer element 282 can be rotated to abrade and cut tissue to cause bleeding when is a collapsed or partly collapsed position. Further, the inner and outer elements 282 and 284 can be patterned with surface electrodes to perform the ablation step. To actuate the implant to an expanded shape as in FIG. 6A, the inner element 284 can be pulled proximally to bend the outer element 284 which can be locked in place by a ratchet mechanism, heat actuated melt adhesion of the elements or any suitable mechanical locking, mechanism. Thus, the implant 275 of FIGS. 6A-6B can again provide the key functions of previous variations, including: flexibility to follow a tortuous path, an RF electrode arrangement to ablate tissue, a cutting mechanism to cause bleeding in a targeted site, means to flatten the fallopian tube and means to trap the coagulum in the targeted site.

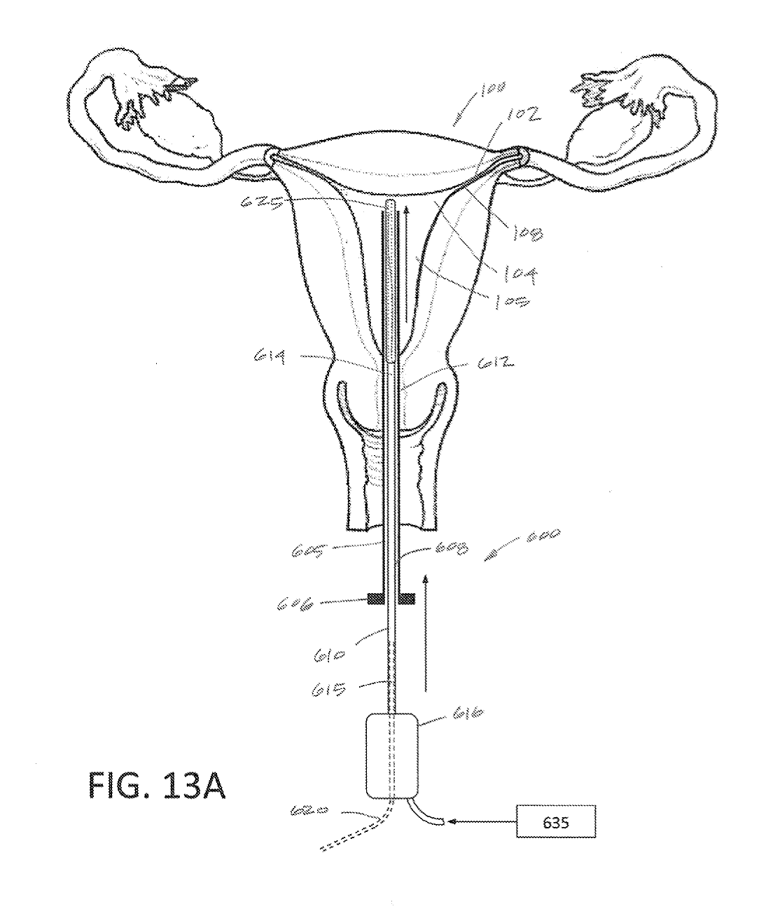

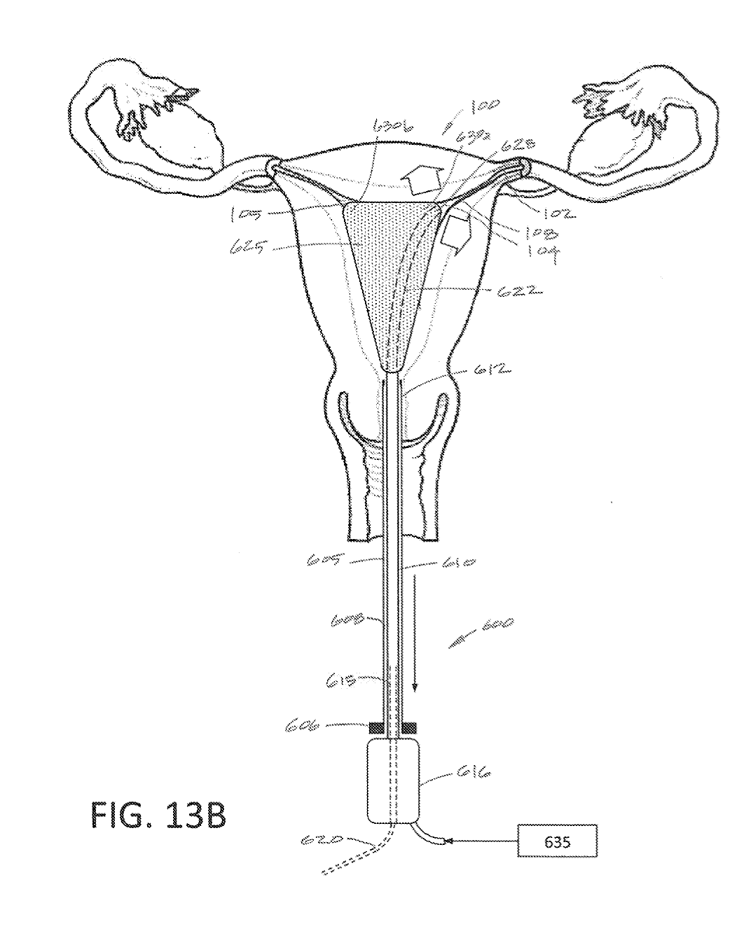

FIGS. 7A-7B depict another variation of implant 325 for use in occluding a fallopian tube that again can function to perform the methods described above. The body 326 of implant 325 has first and second, or respectively, outer and inner polymer sleeve elements 332 and 334 that can be actuated to expand leg elements 335 laterally to flatten the fallopian tube lumen. It can be seen that the outer element has a plurality of slots 340 and the inner element 334 has living-hinged leg elements 335 that can lay flat in the slots 340 in the insertion configuration of FIG. 7A. The inner sleeve 334 can be moved axially relative to outer sleeve 332 over guidewire 342 as shown in FIG. 7B to cause the lea elements 335 to be flexed outwardly. The extended leg elements 335 then will trap blood and coagulum in the site, with the mechanism to cause bleeding described below.

In order to perform the step to cause bleeding in the targeted site in a fallopian tube, the outer sleeve element 332 has a surface 345 covered at least in part with abrasive particles, for example diamond particles or powder bonded to the surface 345. Thus, the outer element 332 can be rotated to abrade and cut tissue to cause bleeding when the implant 325 is in the non expanded position of FIG. 7A. The implant 325 also allows for negative pressure to be applied to the site through the outer sleeve lumen 350 that accommodates the inner sleeve 334. In order to provide the ablation step, the outer surface 345 also can comprise a first polarity electrode with the guidewire 342 comprising the second polarity electrode.

To actuate the implant 325 to an extended or expanded shape of in FIG. 7B, the inner element 334 is pulled proximally to outwardly flex the leg elements 335 which can be locked in place by a ratchet mechanism, heat actuated melt adhesion of the elements or any suitable mechanical locking mechanism. Thus, the implant 325 of FIGS. 7A-7B can again provide the functionality of previous variations, including: flexibility to follow a tortuous path, an RF electrode arrangement to ablate tissue, an abrasive mechanism to cause bleeding in a targeted site, means to flatten the fallopian tube and means to trap the coagulum in the targeted site.

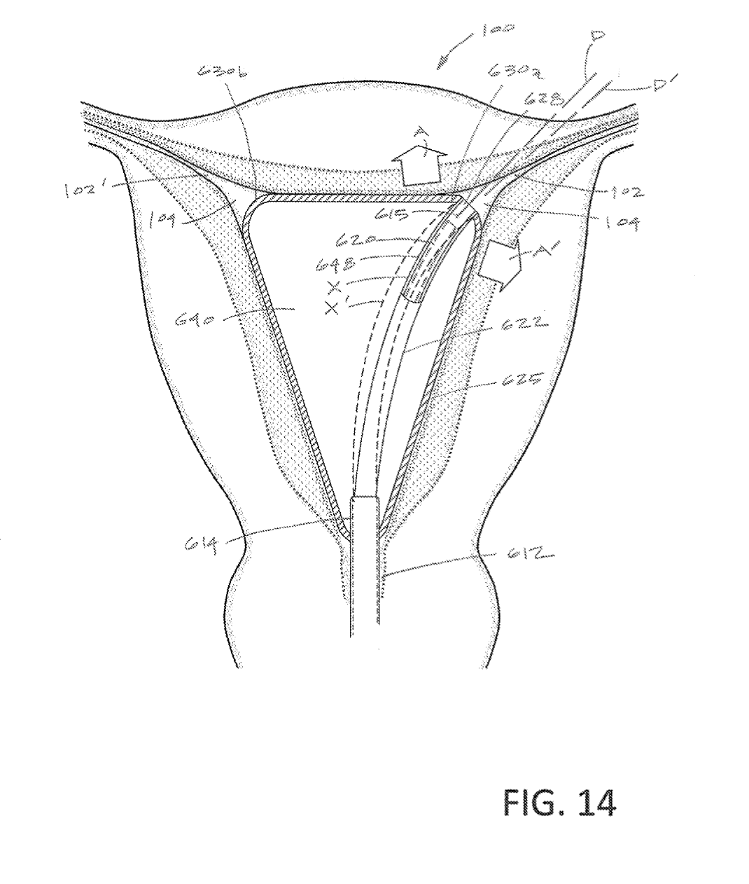

FIG. 8 illustrates a portion of another variation of implant 425 for occluding a fallopian tube that functions to perform methods described previously and is similar to the implant 325 of FIGS. 7A-7B. In FIG. 8, the body 426 of implant 425 has outer and inner polymer sleeve elements 432 and 434 that are actuated to extend leg elements 435 outwardly. In this variation, the leg elements 435 are hollow and needle-like to penetrate tissue and allow bleeding to flow back to site through ports 444 and 445. In other respects, the implant 425 is similar to that of FIGS. 7A-7B with the leg elements 435 being collapsible into a plurality of slots 455. The inner sleeve 434 is moved axially relative to outer sleeve 432 over guidewire 460 and the extended legs 435 then will trap blood and coagulum in the site. The mechanism to cause bleeding is described in the previous embodiment. The outer sleeve element 432 has a surface 465 covered at least in part with abrasive diamond particles bonded to the surface 465. Thus, the outer element 432 can be rotated to abrade and cut tissue to cause bleeding when the implant 425 is in the non-expanded position as in FIG. 8. The outer surface 465 can comprise a first polarity electrode as described previously.

FIGS. 9A-9C illustrate another variation of implant 515 for a fallopian tube that comprises a flexible polymer with multiple flex elements 518 that can flex outwardly to flatten a fallopian tube 102. The flex elements 518 can be resilient, and flex outward as in FIG. 9B after retraction of a retaining sheath (cf. FIG. 1D, 1E and 2B). Alternatively, the flex elements 518 can be flexed by the pull of an inner sleeve in guidewire lumen 520 as shown in the embodiment of FIGS. 6A-6B. The implant 515 can have an abrasive surface 522 for causing bleeding as described previously as well as surface electrodes as described in earlier embodiments.

FIGS. 10A-10B illustrate another variation of implant 525 for occluding a fallopian tube that comprises a polymer with hinged elements 528 that can flex outwardly to flatten a fallopian tube. This embodiment includes barbs 540 for penetrating and gripping tissue. It should be appreciated that all of the previous variations can include barb features for engaging the walls of the fallopian tube. In one variation, an implant can have barbs that point in both the proximal and distal directions to assist in resisting dislodgement when subjected to both peristalsis and antiperistalsis The implant 525 can have an abrasive surface 522 for causing bleeding and surface electrodes as described in earlier embodiments.

FIGS. 11A-11B illustrate another variation of implant 555 for occluding a fallopian tube that has resilient polymer barb elements 558 that Ilex outwardly to grip and flatten a fallopian tube. FIGS. 12A-12B depict another variation of implant 565 that has resilient flex elements 568 that flex outwardly and have barbs 570 facing both proximal and distal directions to engage and flatten a fallopian tube. The variations of FIGS. 11A, 11B, 12A and 12B can include a retractable sheath as described previously as well as surface electrodes as described above.

In some embodiments above, the polymer implants are of a bio-absorble material. Such materials are well known in the art and can be described as bio-resorbable, absorbable bio-erodible and can be assimilated by the body at predictable rates. Bio-resorbable or bio-degradable polymers include polylactic acid (PLA) polyglycolic acid (PGA), polydioxanone (PDS), polyhydroxybutyrate (PHB), polyhydroxyvalerate (PHV), polycaprolactone, polycyanocrylates, or polyphosphazenes. As used herein, the term bio-resorbable includes a suitable bio-compatible material, mixture of materials or partial components of materials being degraded into other generally non-toxic materials by an agent present in biological tissue, for example by being biodegradable or being removed by cellular activity, by bulk or surface degradation, or a combination of one or more of bio-degradable, bio-erodable, or bio-resorbable materials.

FIGS. 13A-13B illustrate another variation of accessing, viewing and navigating a treatment catheter to a targeted location in a fallopian tube. In many cases, a woman's uterus 100 and/or cornua 104 can have an irregular shape or configuration making it difficult to access a fallopian tube 102. Further, the fallopian tube may have a tortuous lumen or a sharp bend in the intramural segment 108 of the tube. FIGS. 13A-13B and 14 schematically depict a device 600 corresponding to invention variation of a device that is adapted to assist introducing an endoscope/treatment catheter/guide wire into a fallopian tube 102.

As can be seen in FIG. 13A, the device 600 includes an introducer sleeve 605 with an optional proximal grip 606 and a lumen 608 that accommodates a guide member 610. FIG. 13A shows the assembly of the sleeve 605 and guide member 610 being introduced through cervical canal 612 into the uterine cavity 105. The guide member 610 has an elongated shaft assembly 614 with guide passageway 615 therein to receive an articulating endoscope 620 and treatment catheter similar to the system shown in FIGS. 1A-1E above. However, any number of devices can be advanced through the passageway 615. The guide passageway 615 extends through handle 616, shaft assembly 614 and flexible guide sleeve 622 (FIG. 13B) in an expandable structure 625 to an open termination 628 (FIG. 13B) that can be adjusted in position and orientation to access the fallopian tube 102 (FIG. 13B). In one variation shown in FIG. 13B, the expandable structure 625 has a triangular shape and can have truncated distal apexes 630a and 630b in which the open termination 628 is disposed. The guide passageway 615 can be sized as needed (e.g., from 3 mm to 8 mm in diameter) and extends through shaft assembly 614 and flexible guide sleeve 622 to the open termination area 628.

FIG. 13B depicts sleeve 605 being retracted in the cervical canal 612 to expose the expandable member 625 carried by the shaft assembly 614. FIG. 13B further depicts the expandable structure 625 being expanded in the uterine cavity 105. In one variation, the expandable structure 625 is inflatable with a fluid (gas or liquid) delivered by a pump 635 such, for example a syringe or squeeze pump. In one example, the inflation pressure can range from 0.25 psi to 5 psi. The expandable structure 625 has interior chamber 640 (FIG. 14) and is configured to occupy a substantial portion of the uterine cavity 105 to form a stable base for a flexible guide sleeve 622 carried within or about the expandable structure 625. In certain variation, the expandable structure has a triangular shape with truncated distal apexes in which the open termination 628 of the guide passageway 615 exits the expandable structure. Other shapes that approximate the shape of the body cavity can be used as well.

Turning, to FIG. 14, a cut-away view of the expandable structure 625 and also shows the guide sleeve 622 in interior chamber 640, wherein the guide sleeve can be a thin-wall flexible polymeric material. An endoscope 620 tin phantom view) is shown being introduced through guide passageway 615. As can be understood from FIG. 14, the expansion of expandable structure 625 can apply a force as indicated by arrow A and A′ which thereby opens the entrance to the fallopian tube 102. The inflation pressure can be adjusted between higher and lower levels to open the cornua 104 either more or less. In one variation, the angle or direction D or D′ at which the endoscope 620 and treatment catheter exit the expandable structure 625 can be adjusted by the physician pushing the shaft assembly 614 slightly back and forth in the cervical canal 612 and uterine cavity 105 to thus alter the orientation of the distal end 648 of the guide sleeve between, for example between shape X and X′ (phantom view). By adjusting the inflation pressure of the expandable structure 625, and by articulating the working end of the endoscope 620, the working channel of the endoscope and the treatment catheter can be aligned with the entrance to an ‘opened-up’ fallopian tube 102. The device 600 will then allow for simplified navigation of the treatment catheter within the fallopian tube 102 as be understood from FIG. 14.

In use, it can be understood that expandable structure 625 can be collapsed and the device can be rotated about its axis by 180° and then expanded to view and access the other fallopian tube 102′.

FIG. 15 shows another variation of device 600′ which includes a first and second flexible guide sleeves 642a and 642b with guide passageways 644a and 644b carried within the expandable structure 625. In one variation, the expandable structure again has a triangular shape with truncated distal apexes 630a and 630b which are configured with the open terminations 648a and 648b of the guide passageways 644a and 644b in the sleeves. The guide sleeves 642a and 642b can be thin polymer tubes that can be flattened to allow for collapse of the sleeves when the expandable structure is collapsed. In one variation, the physician may direct the articulating endoscope 620 into either guide sleeve 642a or 642b as the endoscope is navigated through the expandable structure 625. In another variation, each guide sleeve 642a and 642b can be coupled to a collapsible sleeve that extends back through handle 616 (see FIG. 13B) and thus the physician can then insert the endoscope 620 into either collapsible sleeve at the proximal end of handle 616.

FIG. 16 shows another variation of device 700 that is similar to the device 600 of FIG. 13B. In this embodiment, the expandable structure 725 has first and second inflatable chambers 726a and 726b on either side of flexible guide sleeve 622. In this version, each chamber can be expanded independently allows for adjusting the orientation of the sleeve 622 and opening 628 to align with fallopian tube 102. It should be appreciated that two or more inflatable structures may be positioned about the guide sleeve to open the cornua 104 and fallopian tube 102 as well as aligning the guide passageway 615 with the fallopian tube. In another variation similar to that of FIGS. 13A-13B, an elongate sleeve caring the guide passageway 615 can be axially slidable in the shaft assembly 614, and the elongate sleeve can be moved axially back and forth and torqued from outside the handle 616 to thus flex the sleeve inside the expandable structure to thus align the guide passageway with a fallopian tube.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed is:

1. A method for accessing a fallopian tube, comprising:
trans-cervically introducing an elongate device into a patient's uterine cavity, the elongate device including a flexible guide sleeve having a guide channel with an open distal end;
expanding an expandable member in the uterine cavity to open the uterine cavity and align the guide channel with a fallopian tube.

2. The method of claim 1, wherein the guide sleeve is disposed within the expandable member.

3. The method of claim 1, wherein the guide sleeve is coupled to the expandable member.

4. The method of claim 1, wherein the expanding step comprises inflating the expandable member.

5. The method of claim 1, wherein the expandable member has a triangular shape and the distal termination is proximate an apex of said triangular shape.

6. The method of claim 1, further comprising advancing a catheter through the guide channel and into the fallopian tube.

7. The method of claim 1, where the open distal end of the guide channel opens on a first lateral side of the expandable member.

8. The method of claim 7, where the expandable member comprises a triangular shape and the guide channel opens on a distal apex of the triangular shape.

9. The method of claim 7, further comprising a second flexible guide sleeve having a second guide channel with a second open distal end, where the second open distal end of the second guide channel opens on a second lateral side of the expandable member that is opposite to the first lateral side of the expandable member.

10. The method of claim 8, further comprising adjusting an alignment of the guide channel by adjusting an inflation pressure of the expandable member.

11. The method of claim 8, further comprising adjusting an alignment of the guide channel by deflecting an orientation of the flexible guide sleeve within the expandable member.

17. An access device comprising:
an expandable member comprising a triangular shape having a distal base with a first apex and a second apex on either end of the distal base, and a proximal base opposite to the distal base;
a flexible guide sleeve having a passageway extending therethrough, the flexible guide sleeve extending through the expandable member from the proximal base through to the first apex along the distal base such that the passageway opens at the first apex on a lateral side of the distal base.

13. The access device of claim 12, further comprising an external sleeve exterior to the flexible guide sleeve where the external sleeve and flexible guide sleeve are moveable relative to each other.

14. The access device of claim 12, where a distal end of the flexible guide sleeve is affixed to the expandable member such that a profile of the flexible guide sleeve within the expandable member can be adjusted by relative movement of the flexible guide sleeve to the expandable member.

15. The access device of claim 12, further comprising a second flexible guide sleeve having a second passageway that opens at a second apex on a side of the distal base opposite to the first apex.
